(19) United States
(12) Patent Application Publication
(10) Pub. No.: US 2003/0108495 A1
(43) Pub. Date: Jun. 12, 2003

(54) Cosmetic compositions containing 2,2-dihydroxymethy1-1,3-dioxolanes

(76) Inventor: Eric Matthew Stroud, Oak Ridge, NJ (US)

Correspondence Address: ERIC M. STROUD, 146 NOTCH ROAD, OAK RIDGE, NJ, 07438, US

(21) Appl. No.: 10/263,553

(22) Filed: Oct. 3, 2002

Related U.S. Application Data
(60) Provisional application No. 60/327,161, filed on Oct. 5, 2001.

(51) Int. Cl. A61K 7/42; C07D 323/02

(52) U.S. Cl. 424/59; 549/430

(57) ABSTRACT

Artificial tanning in human skin is induced by 2,2-dihydroxymethyl-1,3-dioxolanes in the presence of water. The 2,2-dihydroxymethyl-1,3-dioxolane is formed by the reaction of 1,3-dihydroxyacetone with a cosmetically acceptable 1,2-diol under anhydrous conditions. The resultant dioloxane is incorporated directly into a cosmetic composition, which is subsequently applied to the skin. The self-tanning reaction rates of 2,2-dihydroxymethyl-4-methyl-1,3-dioxolane and 2,2-dihydroxymethyl-4-ethyl-1,3-dioxolane are approximately equal to 1,3-dihydroxyacetone in water once applied to moistened human skin.

### CROSS REFERENCE TO RELATED APPLICATIONS

This application claims the filing benefit under 35 U.S.C. §119(e) of U.S. Provisional Application 60/327,161, filed Oct. 5, 2001, included herein by reference.

### TECHNICAL FIELD

The present invention is utilizes 2,2-dihydroxymethyl-1,3-dioxolanes produced by the reaction of 1,3-dihydroxyacetone and a specific 1,2-diol. The resultant dioxolane is incorporated into a cosmetic vehicle. Once applied onto moisturized skin, a Maillard reaction browning, or “self-tanning” is initiated, producing an artificial tan.

### BACKGROUND OF THE INVENTION

Most cosmetic and pharmaceutical formulations utilize 1,3-dihydroxyacetone, or “DHA”, to create an artificial tan in human skin. The DHA is typically incorporated into the water phase of a cosmetic composition. The cosmetic composition is applied to moisturized human skin. After approximately five hours after initial application, the Maillard reaction has progressed far enough to produce brown-colored melanoidins in the upper layers of the skin, producing a tan appearance over the application area. This artificial tan remains visible until the upper layers of the skin containing the melanoidins are shed.

The present invention is a unique substitute for DHA dimer or DHA. Using DHA as a starting material, and reacting it with a cosmetically acceptable glycol, a dioxolane is produced. The dioxolane presents alternative methods of incorporation into a cosmetic composition, offers improved handling over DHA dimer powder, yet effectively induces Maillard reaction browning in human skin in the presence of water.

### SUMMARY OF THE INVENTION

Other features and advantages of the present invention will become apparent from the following detailed description, taken in conjunction with the accompanying drawings, which illustrate, by way of example, the principles of the invention.

The present invention is directed to cosmetic compositions containing 2,2-dihydroxymethyl-1,3-dioxolanes for creating an artificial tan in human skin.

The dioxolane is prepared by combining 1,3-DHA dimer with an excess of 1,2-diol, also called a “1,2-glycol”, under anhydrous conditions. The 1,2-diol must be carefully chosen for cosmetic safety and efficacy. For example, 1,2-ethanediol (ethylene glycol), readily forms the cyclic ketal with DHA, but the excess of ethylene glycol presents considerable toxicity risks to humans. 1,2-diols such as 1,2-propanediol (propylene glycol), and 1,2-butanediol (butylene glycol), are much safer and are in common use in cosmetic formulations today.

A vessel containing pure, or nearly anhydrous, 1,2-diol is heat slightly, to approximately 35′-40′ Celsius. All attempts to remove water from the system should be made to achieve the best results. Using low shear slow-speed agitation, for example, a marine propeller blade, 1,3-DHA dimer is added to the 1,2-diol. There should be an excess amount of 1,2-diol present. A molar ratio of 2:1 of diol to DHA is an acceptable ratio. Under constant agitation and constant temperature, the DHA dimer is solvated into the 1,2-diol, resulting in a clear solution of the color of the 1,2-diol used. Mixing times of up to 2 hours are needed to completely solvate the DHA into the 1,2-diol.

Once the solution of 1,2-diol and 1,3-DHA dimer is well mixed, and no dimer particles are seen unsolvated, a small amount of an acid is added to the solution. The solution must now be cooled while agitation is maintained, as the forward reaction with the acid produces heat, which may damage unreacted DHA. An acid should also be chosen with cosmetic acceptability in mind. For example, glacial acetic acid may be a better candidate than hydrochloric acid. Acid is utilized, depending on its strength, at 0.01 to 10% by weight of the mixture. Acid is added slowly to the agitation solution, ensuring even distribution. The solution should be held within the 25-30° C. range once the acid is added, until the reaction is complete.

As the dioxolane or “ketal” reaction progresses, the solution converts from clear to opaque with a marked increase in viscosity. A faint ester-like odor, reminiscent of apples, accompanies certain reaction systems. The resultant product is a waxy solid, white to light brown in color. This waxy solid contains the dioxolane, also known as the “gem diol” or “cyclic ketal” of the 1,3-DHA and 1,2-diol. The product is now stored at room temperature, indefinitely.

When applied directly to moistened human skin or in-vitro models of moistened human skin, the dioxolane product progresses at approximately the safe reaction rate as a DHA/water solution. Brown coloration develops between 2 and 5 hours, depending on the condition of the skin. The dioxolane product may be stored from −5′ C. to 50′ C. over long periods of time without any significant degradation of artificial tan coloration efficacy. The self-tanning reaction rates of dioxolanes formed from 1,2-propanediol and 1,2-butanediol are nearly identical to DHA and water solution. 2,2-dihydroxymethyl-1,3-dioxolanes from 1,2-diols consisting of three of more total carbons are a good substitute for DHA dimer and DHA.

According to one aspect, the invention is the method of producing dioxolanes from 1,3-DHA and a cosmetically acceptable 1,2-diol. According to another aspect, the present invention is the use of 2,2-dihydroxy-methyl-1,3-dioxolanes for self-tanning applications. According to yet another aspect, the present invention involves cosmetic compositions containing of 2,2-dihydroxymethyl-1,3-dioxolanes in order to create artificial tanning in human skin.

Further details of the above-described method and system are set forth below in the discussion of the preferred embodiments, which should be read in conjunction with the accompanying drawings. As will be apparent from the description below, the method and use of the present invention and its variations are suitable for use with any cosmetic composition containing 2,2-dihydroxymethyl-1,3,-dioxolanes.

### BRIEF DESCRIPTION OF THE DRAWINGS

FIG. 1 illustrates the general reaction mechanism for the formation of a 2,2-dihydroxymethyl-1,3-dioxolane.

FIG. 2 describes the structure of 2,2-dihydroxymethyl-4-methyl-1,3-dioxolane formed using 1,3-DHA and 1,2-propanediol.

FIG. 3 details the structure of 2,2-dihydroxymethyl-4-propyl-1,3-dioxolane formed using 1,3-DHA and 1,2-pentanediol.

FIG. 4 illustrates the structure of 2,2-dihydroxymethyl-4-ethenyl-1,3-dioxolane formed using 1,3-DHA and 1,2-butene-2-diol.

FIG. 5 details the manufacturing processes for producing 2,2-dihydroxymethyl-1-3-dioxolanes.

FIG. 6 describes a cosmetic formulation containing 2,2-dihydroxymethyl-4-methyl-1,3-dioxolane.

### DETAILED DESCRIPTION OF THE INVENTION

The instant invention relates to a the method of producing and using 2,2-dihydroxymethyl-1,3-dioxolanes. More particularly, the present invention is directed to, and concerned with, cosmetic compositions containing 2,2-dihydroxymethyl-1,3-dioxolanes for creating artificial tans in human skin.

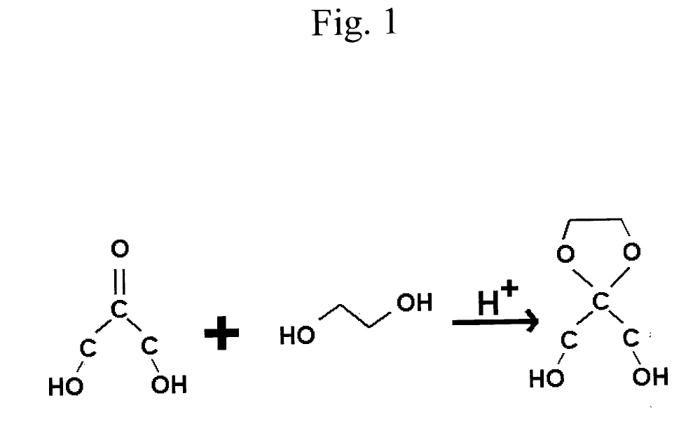

FIG. 1 illustrates the general reaction mechanism for the formation of a 2,2-dihydroxymethyl-1,3-dioxolane. Diols react with ketones under anhydrous conditions to give ketals, which are structurally a dioxolane. The reaction is acid-catalyzed and proceeds most readily when the heterocyclic ring formed is five- or six-membered, therefore, 1,2-diols and 1,3-diols produce the best results. This transformation is used to protect the carbonyl or “ketone” group. In FIG. 1, the carbonyl group of the 1,3-dihydroxyacetone is “protected” once the ketal is formed with 1,2-ethanediol. The result in FIG. 1 is 2,2-dihydroxymethyl-1,3-dioxolane, formed by the ketalization of 1,3-dihydroxyacetone and 1,2-ethandiol under anhydrous conditions in the presence of an acid.

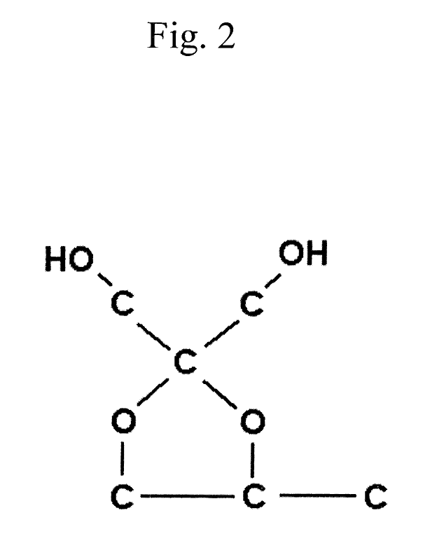

FIG. 2 describes the structure of 2,2-dihydroxymethyl-4-methyl-1,3-dioxolane. The five-membered heterocyclic ring or dioxolane is formed once an excess amount 1,2-propanediol, also called “1,2-propylene glycol” is combined with 1,3-DHA under anhydrous conditions with a small amount of anhydrous acid catalyst.

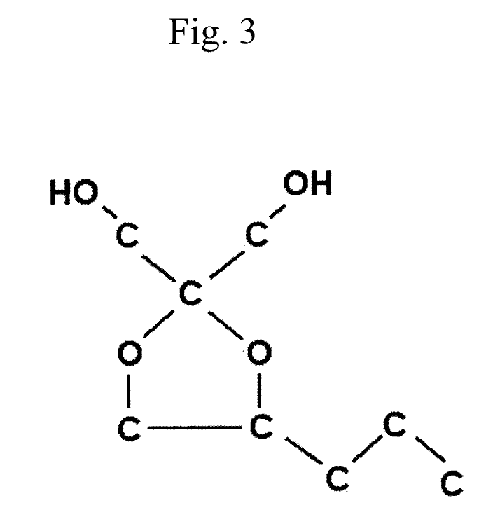

FIG. 3 details the structure of 2,2-dihydroxymethyl-4-propyl-1,3-dioxolane. The five-membered ring or dioxolane is formed once an excess amount 1,2-pentanediol is combined with 1,3-DHA under anhydrous conditions with a d1-10-camphorsulfonic acid catalyst.

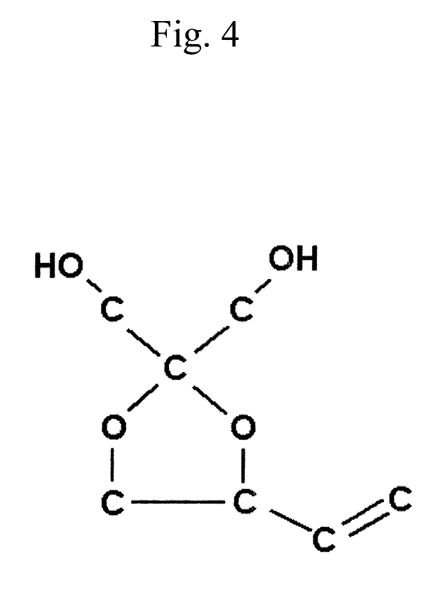

FIG. 4 illustrates the structure of 2,2-dihydroxymethyl-4-ethenyl-1,3-dioxolane formed using 1,3-DHA and 1,2-butene-2-diol. The five-membered ring or dioxolane is formed once an excess amount 1,2-butene-3-diol is combined with 1,3-DHA under anhydrous conditions with an immobilized acid catalyst, also called “IER catalyst”.

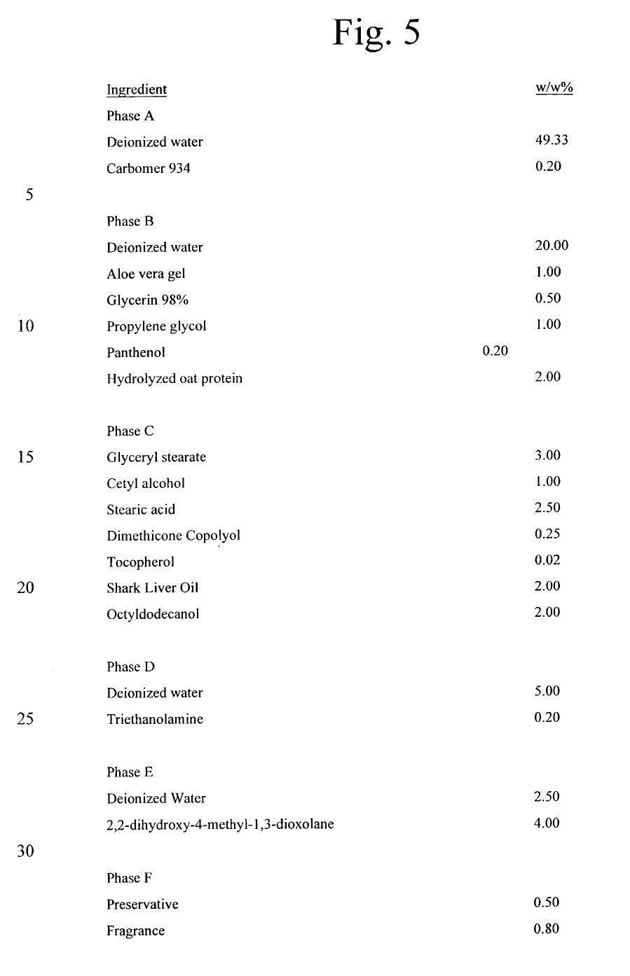

FIG. 5 describes a cosmetic formulation containing 2,2-dihydroxymethyl-4-methyl-1,3-dioxolane. This composition is an oil-in-water emulsion. The formulation is prepared by separately premixing Phases B, C, D, E, and F. Phase E should be premixed using high shear. All of the Phase A water is loaded into a mixing vessel and heating the water to 65′ C. The carbomer is slowly sifted into the heated water with high agitation, and is mixed until uniform. Phase B is added to Phase A under moderate mixing speed. Phase C ingredients are heated and premixed at 78′ C. While Phase A-B is held at 75′ C., Phase C at 78-80′ C. is added to Phase A under moderate mixing. Heat is removed from the batch. When the batch temperature reaches 65′ C., Phase D is added under slow mixing. The batch is cooled to 30′ C., and Phases E and F are slowly added under slow mixing. The cosmetic vehicle is then packaged.

I claim:

1. The use of a 2,2-dihydroxymethyl-1,3-dioxolane for creating an artificial tan in human skin once a 2,2-dihydroxylmethyl-1,3-dioxolane is applied directly to human skin.

2. Said dioxolane in claim 1 is formed by the reaction of 1,3-dihydroxyacetone dimer with a 1,2-diol comprised of at least three carbon atoms

3. Said dioxolane in claim 1 is formed by the reaction of 1,3-dihydroxyacetone dimer with a diol having two adjacent hydroxyl functions.

4. Said dioxolane in claim 1 is formed by the reaction of 1,3-dihydroxyacetone with a diol having two adjacent hydroxyl functions.

5. A cosmetic formulation containing a 2,2-dihydroxymethyl-1,3-dioxolane for creating an artificial tan in human skin the 2,2-dihydroxymethyl-1,3-dioxolane is applied directly to human skin.

6. Said dioxolane claim 5 is formed by the reaction of 1,3-dihydroxyacetone dimer with a 1,2-diol comprised of at least three carbon atoms.

7. Said dioxolane in claim 5 is formed by the reaction of 1,3-dihydroxyacetone dimer with a diol having two adjacent hydroxyl functions.

8. Said dioxolane in claim 5 is formed by the reaction of 1,3-dihydroxyacetone with a diol having two adjacent hydroxyl functions.
